(19) United States
(12) Patent Application Publication
(10) Pub. No.: US 2016/0289169 A1
(43) Pub. Date: Oct. 6, 2016

(54) N-SUBSTITUTED GLYCINIUM BIS(FLUOROSULFONYL)IMIDE IONIC LIQUID

(71) Applicant: CoorsTek Fluorochemicals, Inc., Arvada, CO (US)

(72) Inventors: Rajendra P. Singh, Broomfield, CO (US); Jerry Lynn Martin, Superior, CO (US)

(73) Assignee: CoorsTek Fluorochemicals, Inc., Arvada, CO (US)

(21) Appl. No.: 15/084,425

(22) Filed: Mar. 29, 2016

Related U.S. Application Data
(60) Provisional application No. 62/142,442, filed on Apr. 2, 2015.

(51) Int. Cl. C07C 229/12; C07C 227/40; C09K 3/00; B09C 1/08; C22B 3/44; C11D 7/34; C11D 11/00; C07C 227/18; C22B 7/00

(52) U.S. Cl. CPC C07C 229/12; C07C 227/18; C07C 227/40; C09K 3/00; C22B 7/006; C22B 3/44; C11D 7/34; C11D 11/0029; B09C 1/08

(57) ABSTRACT

The present invention provides N-substituted glycinium bis(fluorosulfonyl)imide compounds and methods for producing and using the same. In particular, the N-substituted glycinium bis(fluorosulfonyl)imide compound is of the formula:

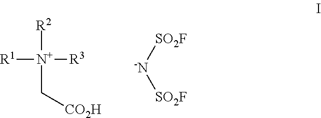

wherein each of R¹, R² and R³ is independently selected from the group consisting of alkyl, aryl, aralkyl, cycloalkyl, (cycloalkyl)alkyl, heteroaryl, (heteroaryl)alkyl, heterocyclyl, and (heterocyclyl)alkyl; or R¹ and R² together with the nitrogen atom to which they are attached to form a nitrogen-heterocyclyl, or a nitrogen-heteroaryl.

### CROSS-REFERENCE TO RELATED APPLICATIONS

This application claims the priority benefit of U.S. Provisional Application No. 62/142,442, filed Apr. 2, 2015, which is incorporated herein by reference in its entirety.

### FIELD OF THE INVENTION

The present invention relates to ionic liquids and a method for producing and using the same. In particular, the present invention relates to N-substituted glycinium bis(fluorosulfonyl)imide ionic liquids and methods for producing and using the same.

### BACKGROUND OF THE INVENTION

Ionic liquids have a wide variety of applications in various fields including, but not limited to, batteries (in particular lithium batteries), removal of metal ions, electrodeposition of metals, and synthesis of nanoparticles. Unfortunately, the solubility of ionic inorganic compounds, e.g., metal salts, are very low in most conventional ionic liquids. This low solubility of metal salts is a serious drawback for possible applications of ionic liquids that require high concentrations of dissolved metal salts, e.g., in lithium batteries, electrodepositions, and synthesis of nanoparticles.

It has been found, however, that ionic liquids comprising a carboxyl group moiety (i.e., —CO₂H), on its cationic moiety portion can increase solubility of metal salts dramatically. See, for example, J. Phys. Chem. B., 2006, 110, 20978-20992 and Inorg. Chem., 2008, 47, 9987-9999. Ionic liquids in these cited references have bis(trifluoromethyl-sulfonyl)imide, i.e., N[(SO₂CF₃)]₂, “TFSI” or “Tf₂N”, as the anionic moiety.

Because of their utility in ability to dissolve or form a complex with inorganic salts, there is a need for other ionic liquids having a carboxyl moiety in its cationic moiety portion.

### SUMMARY OF THE INVENTION

Some aspect of the invention provides an N-substituted glycinium bis(fluorosulfonyl)imide ionic liquid compound of the formula:

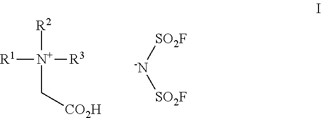

where each of R¹, R² and R³ is independently selected from the group consisting of alkyl, haloalkyl, aryl, aralkyl, cycloalkyl, (cycloalkyl)alkyl, heteroaryl, (heteroaryl)alkyl, heterocyclyl, and (heterocyclyl)alkyl; or R¹ and R² together with the nitrogen atom to which they are attached to form a nitrogen-heterocyclyl, or a nitrogen-heteroaryl.

Another aspect of the invention provides a method for producing an N-substituted glycinium bis(fluorosulfonyl)imide ionic liquid compound of the formula:

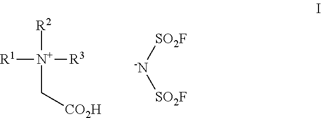

In one embodiment, the method comprises contacting a N-substituted glycinium zwitter-ion of the formula:

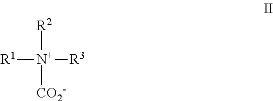

with bis(fluorosulfonyl)imide (i.e., HN(SO₂F)₂ or “HFSI”) under conditions sufficient to produce said N-substituted glycinium bis(fluorosulfonyl)imide ionic liquid compound of Formula I, where R¹, R² and R³ are those defined herein.

Yet in another embodiment, the method comprises contacting an N-substituted glycinium salt of the formula:

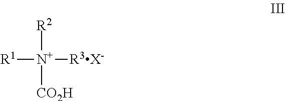

with a metal salt of bis(fluorosulfonyl)imide under conditions sufficient to produce said N-substituted glycinium bis(fluorosulfonyl)imide ionic liquid compound of Formula I, where R¹, R² and R³ are those defined herein.

Still another aspect of the invention provides a method for extracting a metal oxide and/or a metal salt from a sample using a compound of the invention. Typically, the sample comprises a metal oxide, a metal salt or a combination thereof. The method involves contacting the sample with a compound of the invention under conditions sufficient to separate a metal oxide or a metal salt from the sample. Exemplary metal oxides that can be extracted using the method of the invention include, but are not limited to, uranium(VI) oxide, zinc(II) oxide, cadmium(II) oxide, mercury(II) oxide, nickel(II) oxide, copper(II) oxide, palladium(II) oxide, lead(II) oxide, silver(I) oxide, rare earth oxides or a combination thereof.

The compound of invention and the sample are admixed at temperature between 0° C. and 150° C. for extracting the metal oxide and/or the metal salt.

One particular aspects of the invention provides a method for dissolving a metal oxide, a metal hydroxide, a metal salt or a combination thereof from a sample. Such a method comprises contacting a sample that includes a metal hydroxide, a metal salt or a combination thereof with a compound of the invention under conditions sufficient to dissolve a metal oxide, a metal hydroxide, a metal salt or a combination thereof from said sample to produce a solution of dissolved metal comprising said compound of the invention and a processed sample. In this manner, one can decontaminate soil, polish a metal surface, extract precious metal from an ore, etc.

Compounds of the invention are ionic liquids, and therefore can be used in various applications where ionic liquids are used. Such uses are well known to one skilled in the art. Exemplary uses include, but are not limited to, (1) solubilizing organic or inorganic compounds, such as of metal oxides, metal hydroxides or metal salts; (2) extracting processes, e.g., for extracting metal ions from metallurgical slags or from pulverized mixtures of metal oxides with silicate or aluminosilicate rocks; (3) decontamination of soils that are contaminated by heavy metals (especially with copper, nickel, zinc, cadmium, mercury or lead); (4) in catalytic reactions where the compound of the invention serves as a solvent, co-solvent or as a catalyst; (5) electrodeposition of metal ions, e.g., by using the compound of the invention a solvent for the metal precursors; (6) as a medium for electropolishing or for the cleaning of metal surfaces, e.g., by removing metal oxide from its surface; (7) in electrodeposition of (thin) metal layers on conductive surfaces, e.g., by dissolving metal ions and then using electrolysis to deposit the metal layer; (8) processing of spent nuclear fuel elements, e.g., for removing lanthanide or actinide oxides from nuclear fuel elements; (9) as electrolytes in batteries, fuel cells, photovoltaic devices and electrochromic devices; and (10) recycling metals from used catalysts and electronic circuits.

### DESCRIPTION OF THE INVENTION

Definitions:

The term “alkyl” refers to a saturated linear monovalent hydrocarbon moiety of one to twenty, typically one to twelve, and often one to six carbon atoms or a saturated branched monovalent hydrocarbon moiety of three to twenty, typically three to twelve and often three to six carbon atoms. Alkyl groups can optionally be substituted with an alkoxide (i.e., —OR^{a}, where R^{a} is alkyl) and/or other functional group(s) that are either protected or non-reactive under a given reaction condition. In addition, one or more hydrogen atoms of the alkyl group may be replaced by same or different halo atoms. Exemplary alkyl groups include, but are not limited to, methyl, ethyl, n-propyl, 2-propyl, tent-butyl, pentyl, —CH₂Cl, —CF₃, —CH₂CF₃, —CH₂CCl₃, and the like.

The term “cycloalkyl” refers to a non-aromatic, saturated, monovalent mono- or bicyclic hydrocarbon moiety of three to ten ring carbons. The cycloalkyl can be optionally substituted with one or more, for example, one, two or three, substituents within the ring structure that are either protected or unreactive under a given reaction condition. In addition, one or more hydrogen atoms of the cycloalkyl group may be replaced by same or different halogen atoms.

The terms “halo,” “halogen” and “halide” are used interchangeably herein and refer to fluoro, chloro, bromo, or iodo.

The terms “heterocyclyl” and “heterocycloalkyl” are used interchangeably herein and refer to a non-aromatic mono-, bi- or tricyclic moiety of three to twenty, typically three to twelve and often three to eight ring atoms in which one or two ring atoms are heteroatoms selected from N, O, or S(O)ₙ. (where n is an integer from 0 to 2), the remaining ring atoms being C, where one or two C atoms can optionally be a carbonyl group. The heterocyclyl ring can be optionally substituted independently with one or more, preferably one, two, or three, substituents. When two or more substituents are present in a heterocyclyl group, each substituent is independently selected. In addition, one or more hydrogen atoms of the heterocycloalkyl may be replaced by the same or different halogen atoms. Exemplary heterocycloalkyls include, but is not limited to, tetrahydropyranyl, piperidino, piperazino, morpholino and thiomorpholino, thiomorpholino-l-oxide, thiomorpholino-1,1-dioxide, and the like.

The term “aryl” refers to a monovalent mono-, bi- or tricyclic aromatic hydrocarbon moiety of six to twenty, typically six to twelve and often six to ten ring atoms which is substituted independently with one or more substituents. Exemplary aryl includes, but is not limited to, phenyl, 1-naphthyl, and 2-naphthyl, anthracenyl, and the like.

The term “heteroaryl” means a monovalent mono-, bi- or tricyclic aromatic moiety of 5 to 20, typically 5 to 12 and often 5 to 10 ring atoms containing one, two, or three ring heteroatoms selected from N, O, or S, the remaining ring atoms being C. Exemplary heteroaryls include, but is not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, benzodiazepin-2-one-5-yl, and the like.

The terms “(cycloalkyl)alkyl”, “(heterocycloalkyl)alkyl”, “aralkyl”, “(heteroaryl)alkyl” refers to a moiety of the formula —R^{a}R^{b}, where R^{a} is alkenyl and R^{b} is cycloalkkyl, heterocycloalkyl, aryl, and heteroaryl, respectively.

The term “alkenyl” refers to alkyl group as defined herein which is divalent, i.e., having two attaching bonds.

The term “ionic liquid” refers to a salt having a melting point of about 100° C. or less, typically about 80° C. or less, often 50° C. or less, and most often about 25° C. or less.

As used herein, the terms “treating”, “contacting” and “reacting” refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “absence of any solvent” means no solvent is added. While some solvent may be present inadvertently, the total amount of solvent is about 5% or less, typically about 3% or less, often about 1% or less, and most often about 0.5% or less of the total weight of the mixture.

Compounds of the Invention and Synthesis Thereof.

The present invention provides an ionic liquid compound of the formula:

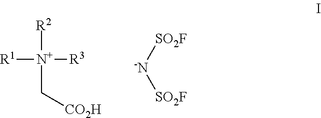

where each of R¹, R² and R³ is independently selected from the group consisting of alkyl, haloalkyl, aryl, aralkyl, cycloalkyl, (cycloalkyl)alkyl, heteroaryl, (heteroaryl)alkyl, heterocyclyl, and (heterocyclyl)alkyl; or R¹ and R² together with the nitrogen atom to which they are attached to form a nitrogen-heterocyclyl, or a nitrogen-heteroaryl.

In some embodiments, each of R¹, R² and R³ is independently alkyl. In one particular embodiment, R¹, R² and R³ are methyl. In other embodiments, at least one of R¹, R² and R³ is methyl. Still in other embodiments, at least two of R¹, R² and R³ are methyl.

Still in another embodiment, R¹ and R² together with the nitrogen atom to which they are attached to form a nitrogen-heterocyclyl or a nitrogen-heteroaryl. In one particular embodiment, R¹ and R² together with the nitrogen atom to which they are attached to form a nitrogen-heterocyclyl or a nitrogen-heteroaryl selected from the group consisting of 3,4-dihydro-2H-pyrrolidinium, 2,3,4,5-tetrahydropyridinium, imidazolium, pyridinium, pyrrolidinium, piperidinium, and morpholinium.

Compound of Formula I can be produced by contacting or reacting an N-substituted glycinium zwitter-ion compound of the formula:

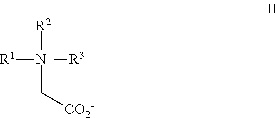

with bis(fluorosulfonyl)imide, i.e., HN(SO₂F)₂ or “HFSI”, under conditions sufficient to produce said ionic liquid compound of Formula I, where R¹, R² and R³ are those defined herein.

The reaction can be conducted in the presence or in the absence of a solvent. If solvent is used, typically water or an alcoholic solvent, such as ethanol, propanol, isopropanol, butanol, etc. or a mixture thereof, is used.

The reaction can be carried out by adding compound of Formula II to HFSI or by adding HFSI to compound of Formula II. Typically, HFSI is added to the zwitter-ion of Formula II slowly to maintain the reaction temperature below a certain temperature, e.g., at or below 70° C., typically at or below 65° C., and often at or below 60° C.

Once the addition is completed, the reaction temperature may be increased to expedite the reaction. The reaction temperature can vary widely and can depend on a variety of factors including, but not limited to, whether a solvent is used or not, the amount of HFSI added, the concentration of the reagents, the reaction time, etc. Typically, when no solvent is used the reaction temperature is increased to at least about 65° C. and often to at least about 70° C. In some embodiments, after complete addition of HFSI, the reaction temperature is increased to about 70° C. When a solvent, e.g., water, is used for the reaction the reaction temperature of about 50° C. or less and often about 40° C. or less is used.

The reaction time can also vary widely depending on various factors including, but not limited to, whether a solvent is used or not, the amount of HF SI added, the concentration of the reagents, temperature of the reaction, etc. In general, however the reaction time is about 2 hours or less, typically about an hour or less, and often about 0.5 h.

In another embodiment, compound of Formula I can be produced by contacting an N-substituted glycinium salt of the formula:

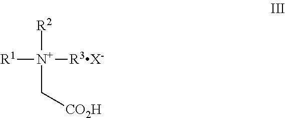

with a salt of bis(fluorosulfonyl)imide under conditions sufficient to produce said compound of Formula I, where R¹, R² and R³ are those defined herein. In salt of Formula III, X⁻ can be any counter ion of a strong acid such as, but not limited to, chloride, bromide, iodide, phosphonate, sulfonate, and the like. A “strong acid” refers to a compound whose pKa is sufficiently low enough to protonate a Compound of Formula II. Typically, a strong acid has pKa of about 5 or less, often pKa of about 3 or less, often pKa of about 1 or less. The term “about” refers to ±20%, typically ±10%, and often ±5% of the numeric value.

Reaction conditions are typically similar to that described above.

Utility

Compounds of the invention have a wide variety of applications that are known to one skilled in the art of ionic liquids. Exemplary application of compounds of the invention includes, but is not limited to, extracting a metal oxide or salt from a sample comprising a metal oxide or salt. Other uses of ionic compounds of the invention include as a solvent in electrochemical applications, e.g., as electrolytes in batteries, in photovoltaic devices, as a medium for electrodeposition, electropolishing of metals, as a solvent for nanoparticle synthesis, and other applications known to one skilled in the art. In addition, since ionic liquids have low vapor pressure and/or high ignition points, they do not generate dangerous air-vapor mixtures. Therefore, they can are particularly useful solvents for chemical reactions, including catalytic reactions.

With regards to extracting a metal oxide or a metal salt using a compound of the invention, such a method typically includes contacting a sample with an ionic liquid compound of Formula I under conditions sufficient to separate a metal oxide from the sample. Exemplary metal oxides that can be extracted using compounds of the invention include, but are not limited to, uranium (VI) oxide, zinc (II) oxide, cadmium (II) oxide, mercury (II) oxide, nickel (II) oxide, copper (II) oxide, palladium (II) oxide, lead (II) oxide, silver (I) oxide, rare earth oxides, or a combination thereof. Exemplary metal salts that can be extracted include CuCl₂.2H₂O or EuCl₃.6H₂O. Often the sample and the compound of Formula I are combined in a solution, typically an aqueous and/or an alcoholic solution.

Extraction of metal oxides or salts can be conducted under a wide range of temperature including, but not limited to, from about 0° C. to about 100° C. Typically, it is conducted at room temperature.

Compounds of the invention have a relatively low melting point compared to other similar ionic liquids, e.g., other N-substituted glycinium ionic liquids such as betaine-bis(trifluoromethylsulfonyl)imide, i.e., “[Hbet][TFSI]”. Typically, the melting point of compounds of the invention is about 100° C. or less, often 80° C. or less, more often 60° C. or less, and most often 50° C. or less.

The viscosity of compounds of the invention also is relatively low compared to other N-substituted glycinium ionic liquids. Typically, the viscosity of compounds of the invention is at least 5% lower, often at least 10% lower and more often at least 20% lower than the viscosity of other known N-substituted glycinium ionic liquids, such as [Hbet][TFSI]. Low viscosity is advantageous, as it allows increased mass transfer and more rapid metals extraction.

As stated above, compounds of the invention can be used in a variety of application. In one particular embodiment, compounds of the invention are used to solubilize metal substrates. Exemplary metal substrates that can be solubilized by compounds of the invention include, but are not limited to, metal oxides, metal hydroxides, metal salts, etc.

Compounds of the invention can be used to dissolve metal oxides such as: Sc₂O₃, Y₂O₃, U₂O₃, Pr₆O₁₁, Nd₂O₃, Sm₂O₃, Eu₂O₃, Gd₂O₃, Tb₄O₇, Dy₂O₃, Ho₂O₃, Er₂O₃, Tm₂O₃, Yb₂O₃, Lu₂O₃, UO₃, PbO, ZnO, CdO, HgO, CuO, Ag₂O, NiO, PdO and MnO. Similarly, compounds of the invention can also be used to dissolve hydroxides of these metal oxides. In this manner, compounds of the invention can be used in chemical reaction where use of a non-protic solvent is desired or required. In some cases, water can be used to facilitate the dissolution of the metal oxide or metal hydroxide, after which water can be removed. Metal salts can also be dissolved by compounds of the invention in a similar manner with or without the aid of water. Exemplary metal salts that can be dissolved include, but are not limited to, CuCl₂.2H₂O and EuCl₃.OH₂O.

The dissolution process can be facilitated by using moderate to high pressure conditions.

The metals that are dissolved using compounds of the invention can be recovered or removed by extracting the ionic liquid of the invention with an acidified aqueous solution such as a dilute hydrochloric acid or nitric acid solution. Such extraction transfers the metals to the aqueous phase and the compound of the invention is regenerated. In this manner, a sample (e.g., soil, or other material) can be decontaminated using compounds of the invention. A metal surface can also be cleaned using a similar process to remove the metal oxide coating from its surface. Thus, one particular use of compounds of the invention include recycling precious metals, e.g., platinum, from catalysts and the electrodeposition process.

Compounds of the invention can also be used in ore processing. In particular, extracting precious metals from ores. Due to its selective solubility of metal oxides, compounds of the invention have advantage for the extraction for metals from ores. For example, precious or valuable metals can be extracted from ores whereas other non-metal materials, such as the quartz, silicates, aluminosilicates, aluminum oxides and iron oxides are unaffected and can be readily separated.

Compounds of the invention can also be used in metal processing. For example, by dissolving metals (e.g., from metal salts), one can electrodeposits metals on to other surfaces using electrolysis. Compounds of the invention can also be used in electroplating using a similar process. Metal surfaces can also be polished by removing metal oxide coating from the surface using compounds of the invention.

Because compounds of invention are ionic liquids, they can also be used in electrolytes for batteries, fuel cells and photovoltaic cells. As such, compounds of the invention are particularly useful in lithium batteries.

Selective dissolving of lanthanide and actinide series of metals also allows compounds of the invention to be used in processing spent nuclear fuel elements. In addition, selective dissolving properties of heavy metals allow compounds of the invention to be used in cleaning or decontamination of soils as well as in recovery of various metals such as copper, zinc and lead from a wide variety of samples.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Unless otherwise stated, all the chemicals used were of reagent grades. Bis(fluorosulfonyl)imide (i.e., HN(SO₂F)₂) was prepared as reported. See, for example, commonly assigned U.S. Pat. No. 8,377,406, issued Feb. 19, 2013, which is incorporated herein by reference in its entirety.

### EXAMPLE 1

This example illustrates a process for producing neat protonated trimethylglycinium bis(fluorosulfonyl)imide (“TMG FSI”). This compound may also be referred to as betaine bis(fluorosulfonyl)imide or betainium bis(fluorosulfonyl)imide (“hbet FSP”).

In a 100 ml dry flask equipped with stirring device, bis(fluorosulfonyl)imide (18.1 g, 0.1 mol) was mixed with betaine powder (11.7 g, 0.1 mol) at room temperature under argon atmosphere. Betaine was added slowly in such a way that the temperature in the flask did not reach >60° C. After complete addition of betaine, the resulting reaction mixture was heated to about 70° C. for 0.5 h. The reaction flask was then evacuated at 50° C. (<100 mtorr) for 1 h, and cooled to room temperature to obtain a colorless viscous liquid. Yield: 29.6 g, 99%.

### Example 2

This example illustrates another process for producing neat trimethylglycinium bis(fluorosulfonyl)imide (“TMG F SI”).

In a 100 ml dry flask equipped with stirring device, bis(fluorosulfonyl)imide (36.2 g, 0.2 mol) was heated with oil bath at 60° C. under argon atmosphere. To this heated material was slowly added betaine powder (23.4 g, 0.2 mol) to maintain the reaction temperature of less than 65° C. After complete addition of betaine, the reaction mixture was heated to about 70° C. for 0.5 h. The reaction flask was then evacuated at 50° C./<100 mtorr for 1 h, and cooled to room temperature to obtain a colorless viscous liquid. Yield: 59.2 g, 99%.

### Example 3

This example illustrates a process for producing trimethylglycinium bis(fluorosulfonyl)imide (“TMG FSI”) using a solvent.

In a 250 ml flask equipped with stirring device, betaine (35.1 g, 0.3 mol) was dissolved in 75 g of deionized water. Neat bis(fluorosulfonyl)imide (54.3 g, 0.3 mol) was added drop wise into the stirring betaine solution while maintaining the reaction temperature of less than 40° C. After complete addition of HFSI, the reaction mixture was stirred for additional 0.5 h. Water was removed on a rotary evaporator at 40° C. and further concentrated at 50 ° C./<100 mtorr for 2 h. Cooling the resulting product to room temperature provided a colorless viscous liquid. Yield: 88.5 g, 99%.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. An ionic liquid compound of the formula: 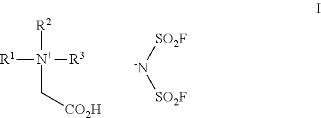
wherein
each of R¹, R² and R³ is independently selected from the group consisting of alkyl, aryl, aralkyl, cycloalkyl, (cycloalkyl)alkyl, heteroaryl, (heteroaryl)alkyl, heterocyclyl, and (heterocyclyl)alkyl; or
R¹ and R² together with the nitrogen atom to which they are attached to form a nitrogen-heterocyclyl, or a nitrogen-heteroaryl.

2. The ionic liquid compound according to claim 1, wherein each of R¹, R² and R³ is independently alkyl.

3. The ionic liquid compound according to claim 1, wherein R¹ and R² together with the nitrogen atom to which they are attached to form a nitrogen-heterocyclyl or a nitrogen-heteroaryl.

4. The ionic liquid compound according to claim 3, wherein R¹ and R² together with the nitrogen atom to which they are attached to form a nitrogen-heterocyclyl or a nitrogen-heteroaryl selected from the group consisting of 3,4-dihydro-2H-pyrrolidinium, 2,3,4,5-tetrahydropyridinium, imidazolium, pyridinium, pyrrolidinium, piperidinium, and morpholinium.

5. A method for producing an ionic liquid compound of the formula: 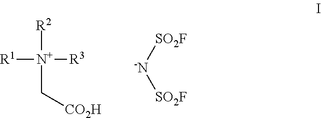
said method comprising:
(a) contacting a zwitter-ion of the formula: 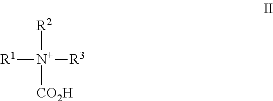
with bis(fluorosulfonyl)imide under conditions sufficient to produce said ionic liquid compound of Formula I; or
(b) contacting a salt of the formula: 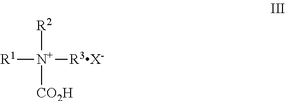
with a metal salt of bis(fluorosulfonyl)imide under conditions sufficient to produce said ionic liquid compound of Formula I;
wherein
each of R¹, R² and R³ is independently selected from the group consisting of alkyl, aryl, aralkyl, cycloalkyl, (cycloalkyl)alkyl, heteroaryl, (heteroaryl)alkyl, heterocyclyl, and (heterocyclyl)alkyl; or
R¹ and R² together with the nitrogen atom to which they are attached to form a nitrogen-heterocyclyl, or a nitrogen-heteroaryl.

6. The method of claim 5, wherein said method comprises anhydrous conditions.

7. The method of claim 5, wherein each of R¹, R² and R³ is independently C₁-C₁₀ alkyl.

8. The method of claim 7, wherein R¹, R² and R³ are methyl.

9. The method of claim 5, wherein said method comprises adding bis(fluorosulfonyl)imide to said zwitter-ion of Formula II while maintaining the reaction temperature below 65° C.

10. The method of claim 9, wherein bis(fluorosulfonyl)imide is added to said zwitter-ion of Formula II at a temperature of at least 50° C.

11. The method of claim 9 further comprising the step of increasing the reaction temperature to above 65° C. after complete addition of bis(fluorosulfonyl)imide.

12. The method of claim 9, wherein said method is conducted in the absence of any solvent.

13. The method of claim 9, wherein said zwitter-ion of Formula II is in an aqueous solution.

14. The method of claim 13, wherein bis(fluorosulfonyl)imide is added to said aqueous solution of zwitter-ion of Formula II at a temperature of 40° C. or less.

15. The method of claim 13, wherein the reaction temperature is maintained at 40° C. or less.

16. The method of claim 15 further comprising the step of removing water while maintaining the temperature at 50° C. or below to obtain said ionic liquid compound of Formula I with a yield of at least 95% having at least 95% purity.

17. The method of claim 5, wherein said salt of Formula III comprises a hydro-halogen salt of Formula III.

18. The method of claim 5, wherein said metal salt of bis(fluorosulfonyl)imide is of the formula: M[N(SO₂F)₂]_{y}, wherein
M is an alkali metal, an alkaline earth metal, or a transition metal; and
y is an oxidation state of M.

19. The method of claim 18, wherein said the reaction between said salt of Formula III and said salt of bis(fluorosulfonyl)imide is conducted in the presence of a solvent.

20. The method of claim 19 further comprising the step of removing M(X)_{y} from the reaction mixture, wherein y is as defined in claim 18.

21. A method for dissolving a metal oxide, a metal hydroxide, a metal salt or a combination thereof from a sample, said method comprising contacting a sample that comprises a metal hydroxide, a metal salt or a combination thereof with a compound of claim 1 under conditions sufficient to dissolve a metal oxide, a metal hydroxide, a metal salt or a combination thereof from said sample to produce a solution of dissolved metal comprising said compound of claim 1 and a processed sample.

22. The method of claim 21, wherein said processed sample comprises decontaminated soil.

23. The method of claim 21, wherein said processed sample comprises a polished metal surface.

24. The method of claim 21, wherein said processed sample comprises a process ore.

25. The method of claim 24 further comprising the step of recovering a precious metal from said solution of dissolved metal.
